(19) United States
(12) Patent Application Publication
(10) Pub. No.: US 2008/0307907 A1
(43) Pub. Date: Dec. 18, 2008

(54) Method for the Prediction of Consumer Acceptance of Food Containing Oils

(76) Inventor: Beat Fluehmann, Zuerich (CH)

Correspondence Address: NIXON & VANDERHYE, PC, 901 NORTH GLEBE ROAD, 11TH FLOOR, ARLINGTON, VA, 22203, US

(21) Appl. No.: 12/097,763

(22) PCT Filed: Dec. 15, 2006
(86) PCT No.: PCT/EP06/12109

§ 371 Date: Aug. 19, 2008

(30) Foreign Application Priority Data
Dec. 28, 2005 (EP) 05028616.0

(51) Int. Cl. G01N 33/03

(52) U.S. Cl. 73/865.7

(57) ABSTRACT

A method for predicting (a) the seriousness of side effects to be expected in humans after consumption of foods or nutritional supplements containing oils rich in polyunsaturated fatty acids and/or (b) the acceptance of such foods or nutritional supplements by consumers, characterized by analyzing the foods or nutritional supplements with automated solid phase micro extraction (SPME) followed by ammonia negative chemical ionization mass spectrometric detection.

The present invention relates to the use of the FAST Index™ for predicting (a) the seriousness of side effects to be expected in humans after consumption of foods or nutritional supplements containing oils and/or (b) the acceptance of such foods or nutritional supplements by consumers wherein the oils are particularly rich in polyunsaturated fatty acids (PUFAs).

During the last years oils containing PUFAs, in the form of glycerides or other esters, especially marine oils, have attracted substantial interest as a source of such PUFAs and have gained increased importance as dietary supplements. Today there is reasonable evidence that increasing the dietary levels of PUFAs has beneficial effects on health and can reduce the incidence of death, e.g., from coronary heart diseases via effects on blood pressure, atherosclerosis, and thrombogenesis. Other beneficial effects on health have been shown or at least been made plausible. This has triggered an increased demand for such oils or for food and food supplements containing them by consumers.

On the other hand with increasing number of double bonds the PUFAs are subject to increasing oxidative degradation and development of undesirable “off-flavors”, mainly fishy smell and taste, which are a limiting factor in consumer acceptance of products containing such PUFAs. The increasing interest in the PUFAs, such as eicosapentaenoic acid (EPA) and docosahexaenoic acid (DHA), has promoted development of methods of refining and stabilizing such oils and concentrates of PUFA esters.

At the same time methods have been developed of rating the taste of such PUFA containing oils by determining quantitatively a certain number of degradation products responsible for the fishy smell and taste of a product, viz. mainly aldehydes and ketones, comparing the profile obtained with a standard profile and assigning a taste factor from 1 to 5 (corresponding to “no fish taste” to “very strong fish taste”, respectively) to the product (see, e.g., U.S. Pat. No. 6,623,774).

Another, highly sophisticated analytical technique has been developed by DSM Nutritional Products (DNP) for characterizing the fishy taste and smell of oils or products containing oils, know as the FAST Index™. The index correlates exactly with trained taste panels to give a scale for fishy-ness. The acronym “FAST” stands for “Fatty Acid Smell and Taste”. The method combines automated solid phase micro extraction (SPME) of smell molecules and ammonia negative chemical ionization mass spectrometric detection. This makes it possible to measure the concentrations of 3 specific molecules (4-heptenal, 2.6-nonadienal and 3.6-nonadienal) which confer fishy taste and smell. An algorithm converts the data into a score which reflects these concentrations. The FAST Index™ has been calibrated with human taste panels such that a score of 1-7 reflects the range of taste sensitivity experienced by human subjects. A score of 1 indicates a complete lack of fishy taste (i.e. “not”), a score of 2 “very slightly”, a score of 3 “slightly”, a score of 4 “middle”, a score of 5 “strong”, a score of 6 “very strong”, whereas a score of 7 indicates an extreme fishy taste. Although the human taste sensation is saturated at a score of 7 or little above, the FAST Index™ can measure taste and smell molecules up to a score of several hundred. In the journal inform (12, 244-249, March 2001) of the American Oil Chemists Society N. Macfarlane et al. have called for a test to quantify fish flavor, described the FAST Index™ technique and disclosed the algorithm by which the analytical data is converted into the score.

Recently a new and surprising use was discovered for the FAST Index™. It has been shown that the FAST Index™ can be used to predict consumer acceptance of nutritional supplement or food products enriched with oils, especially rich in PUFAs, even after the product has been ingested by the consumer. This application of the FAST Index™ is a powerful tool for developing and testing PUFA-containing end products which are acceptable to consumers, hence improving consumer satisfaction and safeguarding the consumer from unpalatable or unpleasant products. This discovery was made by using the FAST Index™ to analyse data obtained from a trial on consumer acceptance of nutritional supplements containing fish oils. The double-blind, placebo controlled, cross over consumer study, presented at the American Oil Chemist's Society conference May 9-12, 2004, was performed to test acceptance of a number of soft gelatine capsules containing different fish oil-derived long chain PUFAs (LC-PUFAs) in form of their esters (i.e. ethyl esters and/or glycerides). Consumers were asked to take the capsules or a placebo and report on negative side effects in the hours following ingestion. The consumers were not aware of which product they were taking. In the case of three of the four fish oil products, severe and numerous side effects were reported, notably belching with strong fishy aftertaste, starting approximately one hour after ingestion. However, significantly fewer side effects than from other products were reported for fish oil capsules, containing ROPUFA® 75% n-3 EE which is a refined marine oil with minimum content of 72% n-3 PUFAs in form of ethyl esters [DHA:EPA=22:42] and which is stabilized with mixed tocopherols, ascorbyl palmitate, citric acid and contains rosemary extract. The ROPUFA® 75% n-3 EE is obtainable from DSM. The study also demonstrated a higher purchase intent associated with the fish oil capsules containing the ROPUFA® 75% n-3 EE.

Subsequent to the above mentioned study the number of side effects experienced for each product was plotted against the FAST Index™ of the products. There was a good correlation between the FAST Index™ score and the number of reported side effects arising from a given capsule type. This was surprising and unexpected in view of the fact that the capsules had already been ingested. It is the first time that a fishy taste and smell phenomenon arising after ingestion of a product has been directly related to an analytical result obtained using the FAST Index™ from the oil prior to ingestion. The data obtained clearly demonstrates that the higher the FAST Index™, the higher the number and/or seriousness of side effects. This was an unexpected finding because the side effects are noted after the product has been ingested, during which time digestion in the stomach has been initiated. Despite the fact that the product has been ingested, the FAST Index™ is a good predictor of the later side effects which can be expected from a given products. In contrast traditional chemical markers of fish oil quality, such as peroxide value or para anisidine value, do not correlate with the sensory quality of an edible oil. The FAST Index™ is a useful tool for assessing consumer acceptance and satisfaction of products containing PUFAs by predicting the frequency/severity of side effects even after ingestion of the product. It is a new quality parameter.

Therefore the present invention relates to a method for determining the seriousness of side effects to be expected in humans after consumption of foods or nutritional supplements containing oils which method is characterized by analyzing the foods or nutritional supplements using the FAST Index™ methodology and evaluating the result with the Fish Taste algorithm as well as to a method for predicting the acceptance of foods or nutritional supplements containing oils by consumers which method is characterized by analyzing the food or nutritional supplement oils using the FAST Index™ methodology and evaluating the result with the Fish Taste algorithm. Typical side effects of oils or food containing oils after ingestion are fishy reflux and fishy after taste.

The term “seriousness of side effects” comprises both the degree of a specific side effect as well as the frequency of different side effects.

The term “oil” relates to oils suitable for ingestion by humans and animals, preferably to oils derived from microorganisms, plants or animals, in diluted or concentrated form, obtained by all known methods, with or without purification and stabilization. Examples of such oils are oils from plants like rape, flax, borage, evening primrose, from fish like tuna, herring, sardine, anchovy or from algae. Preferred oils are those which are rich in PUFAs.

The oil may be present as such, i.e., as a liquid, emulsion or in microencapsulated form for a better handling and further processing or stabilization.

The term “food” comprises all kinds of foods including animal feed (including pet food) and beverages, prepared and/or processed by any method known in the art.

The term “nutritional supplement” comprises all kinds of compounds and mixtures thereof which are used to enrich food and feed products with physiological valuable components thus increasing their nutritional or health value.

The terms “PUFA” and “LC-PUFA” are used in their generally accepted meanings; they relate to fatty acids with at least 2 carbon-carbon double bonds, preferably consisting of 18-22 carbon atoms, and comprise n-3, n-6 and n-9 acids. Although the term PUFA defines free acids it is generally understood to also mean their salts and these acids in the form of their naturally occurring esters, i.e. as glycerides (comprising mono-, di- and triglycerides) and in form of esters into which they are converted, e.g. by transesterification, such as ethyl esters. PUFAs of preferred interest in the context of the present invention are n-3 and n-6 PUFAs, espec. EPA, DPA, DHA, GLA and ARA, preferably of food-grade quality, as single compounds or in mixtures, preferably in the form of their esters, e.g., triglycerides, or ethyl esters, especially as components of oils obtained from marine animals, preferably from fish, from plants or by fermentation. They can be stabilized and/or deodorized by methods known in the art, e.g., by addition of antioxidants, emulsifiers, spices or herbs, such as rosemary or sage extracts. In a preferred embodiment of the present invention the term PUFA refers to refined fish oils commercially available and known under the trade mark ROPUFA®. In a further preferred embodiment of the present invention the ROPUFA® has been stabilized with tocopherols or tocotrienols (natural mixtures or synthetically prepared, preferably α-tocopherol), if desired together with other antioxidants and/or deodorants, such as ascorbyl palmitate and/or rosemary extract.

The Fish Taste algorithm is represented by the following equation

FAST Index=1+(0.31A)+(0.1B)+(0.03C)

wherein A, B and C are the concentrations, in ppb, of 2,6-nonadienal, 4-heptenal and 3,6-nonadienal, respectively.

Hereinafter the general detailed description for carrying out the FAST index methodology on oils by HS-SPME-GC-MS is given.

### Principle

Solid phase micro-extraction is used to sample the volatiles present in the headspace of an oil. A small number of key compounds are then quantitatively analysed by GC/MS. Previous work has characterised these compounds in particular as being good indicators of oil oxidation.

### Reagents

trans-2-hexenal
trans, cis-2,6-nonadienal
cis-4-Heptenal
trans, trans-2,4-Heptadienal
1-Penten-3-one
Methyl hexanoate
Ethyl heptanoate

### Instrumentation and Analytical Conditions

### Instrument

Agilent 6890 Plus and 5973 MSD GCMS System, with a CTC CombiPAL, equipped for SPME.

### Column

J&W DB-FFAP, 30 m×0.25 mm×0.25 μm

### GC

Column Oven Programme: 40° C., hold 10 minutes, ramp to 140° C. at 5° C./min, ramp to 240° C. at 100° C./min, hold 19 minutes. Total programme time: 50 minutes.
Carrier Gas: Helium, Constant Pressure at a nominal 14.00 psi. The actual pressure is subsequently determined by Retention Time Locking (RTL) of the method. The GC method is Retention Time Locked to 4-Heptenal at 13.80 minutes.
Injector: Split/Splitless injector with 0.75 mm ID SPME Injection Liner and a High Pressure Merlin Microseal Septum.
Injector Programme: 250° C. isothermal, Splitless for 3 minutes.

### MSD

Negative Chemical Ionisation (NCI), using Ammonia as the reagent gas, operated in Single Ion Monitoring (SIM) mode.
Source Temperature: 150° C.
Quad Temperature: 150° C.
GC/MSD Interface Temperature: 250° C.

### SPME

Fibre: StableFlex Divinylbenzene/Carboxen/Polydimethylsiloxane
(DVB/CAR/PDMS), 50/30 um, Standard Needle with a 1 cm Fibre.
Adsorb in the headspace without agitation at 40° C. for 45 minutes, desorb for 3 minutes, one sampling per vial. Fibres are cleaned by further desorption for 30 minutes at 270° C. in the Fibre Conditioning Accessory.
New Fibres are conditioned for a minimum of 4 hours at 270° C.

### Standard & Sample Preparation and Analysis

### Standard Preparation and Analysis

An internal standard solution is to be prepared in deodorised Miglyol at a nominal concentration of 750 ppm each of methyl hexanoate and ethyl heptanoate. This solution is then to be used as detailed above to provide approximately a 10 ppb concentration of both methyl hexanoate and ethyl heptanoate. External standards solutions are to be prepared in deodorised Miglyol to yield solutions over the concentration range 1 to 100 ppb each of 1-penten-3-one, 2-hexenal, 4-heptenal, 2,4-heptadienal and 2,6-nonadienal.

The Microsoft Excel spreadsheet entitled ‘R&D FAST Cali Calcs_New Calibration.xls’ is to be used to calculate the weights and volumes of standards and Miglyol, respectively, required in order to prepare the solutions described above.

To prepare a standard for analysis, accurately weight 1 g of the appropriate external standard solution into a 10 ml autosampler vial. Tare the balance and carefully add one drop of the internal standard solution using a 150 mm Pasteur pipette. Accurately record the weight of the addition. Cap the vial immediately with a magnetic autosampler vial cap.

Using the PAL Sequence Manager window of the Agilent Chemstation software insert the required number of entries into the sample list. Select the relevant GC/MS acquisition method and the relevant Autosampler method. Complete the remaining fields of the sample list and start the acquisition.

In the event of the instrument having been idle then a blank vial is to be analysed prior to the analysis of standards. This ensures the fibre is free from any molecules, which may have absorbed through exposure to the laboratory atmosphere. A blank vial must also be analysed prior to the analysis of a 1 ppb external standard, and subsequent to the analysis of a 100 ppb external standard.

### Sample Preparation and Analysis

Accurately weight 1 g of oil into a 10 ml autosampler vial. Tare the balance and carefully add one drop of the internal standard solution using a 150 mm Pasteur pipette. Accurately record the weight of the addition. Cap the vial immediately with a magnetic autosampler vial cap.

Using the PAL Sequence Manager window of the Agilent Chemstation software insert the required number of entries into the sample list. Select the relevant GC/MS acquisition method and the relevant Autosampler method, complete the remaining fields of the sample list and start the acquisition.

In the event of the instrument having been idle then a blank vial is to be analysed prior to the analysis of samples. This ensures the fibre is free from any molecules, which may have absorbed through exposure to the laboratory atmosphere. Each sequence of samples must contain at least two standards, usually a 5 ppb and a 10 ppb, for quality control checking.

### Interpretation of Data

The mass chromatograms generated by the GC/MS analyses are to be handled by the Enhanced Data Analysis window of the Agilent Chemstation software. Using the software obtain peak areas for the analytes. In each case, it is important that the peak identity is confirmed both by the presence of the correct m/z ratio and retention time, as detailed below.

| | Molecular | Molecular | Quantification | Retention |
|---|---|---|---|---|
| Compound | Formula | Weight | Ion, m/z | Time, min |
| 1-Penten-3-one | C₅H₈O | 84 | 83 | 3.90 |
| Methyl hexanoate | C₇H₁₄O₂ | 130 | 129 | 10.85 |
| 2-Hexenal | C₆H₁₀O | 98 | 97 | 12.36 |
| 4-Heptenal | C₇H₁₂O | 112 | 111 | 13.80 |
| Ethyl heptanoate | C₉H₁₈O₂ | 158 | 157 | 17.79 |
| 2,4-Heptadienal | C₇H₁₆O | 110 | 109 | 22.82 |
| 3,6-Nonadienal | C₉H₁₄O | 138 | 137 | 25.11 |
| 2,6-Nonadienal | C₉H₁₄O | 138 | 137 | 25.43 |

The areas are to be input into the Microsoft Excel spreadsheet entitled “R&D FAST Cali Calcs_ddmmyy_AnalaysedDDMMYY.xls”

Where ddmmyy is the date the [internal and external] standards were prepared and DDMMYY is the date the standards were analysed and subsequently used to recalibrate the method.

This Excel spreadsheet will covert the individual response areas into concentrations in parts per billion (ppb) for each of the key compounds. It will also simultaneously generate a value for FAST index taste prediction based on the statistically derived formula below:

FAST Index Taste Prediction=1+(0.31A)+(0.11B)+(0.03C)

where A, B and C are the concentrations, in ppb, of 2,6-nonadienal, 4-heptenal, and 3,6-nonadienal, respectively.

At present the concentrations of 1-penten-3-one, 2-hexenal and 2,4-heptadienal are not utilised in the calculation of a FAST index taste prediction However, the concentrations of these three components are to be determined, with a view to incorporating one or more at a later date should there be sufficient statistical data to support this.

The following figures are added:

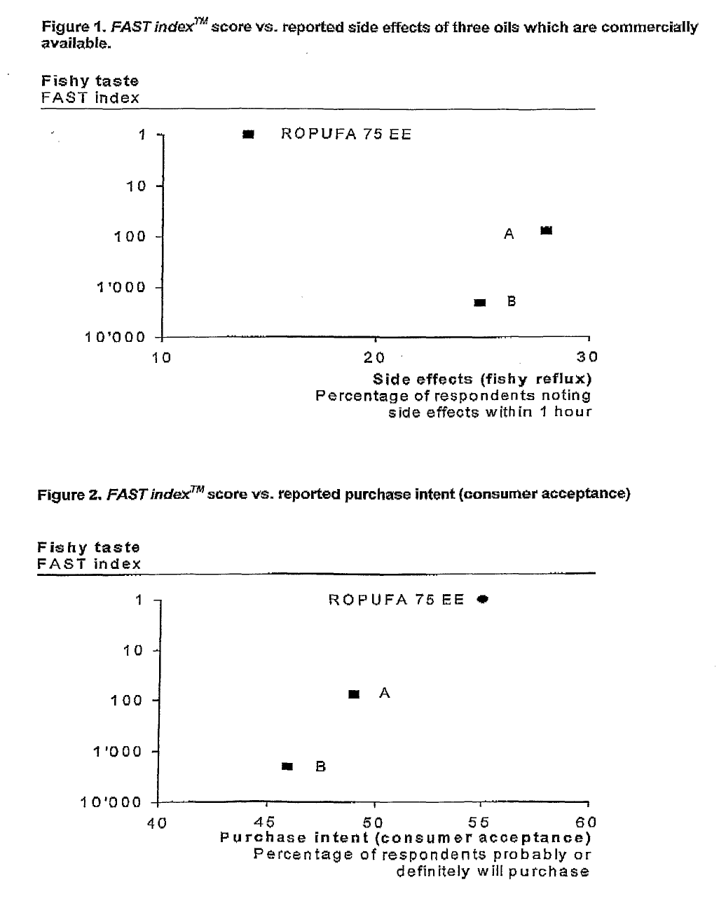

FIG. 1: FAST Index™ score vs. reported side effects of three oils which are commercially available.

FIG. 2: FAST Index™ score vs. reported purchase intent (consumer acceptance).

A represents an oil containing EPA (42%) and DHA (22%) in ethyl ester form.

B represents an oil containing 60% EPA+DHA in ethyl ester form.

ROPUFA® 75 N-3 EE Oil is a refined marine oil with minimum content of 72% n-3 PUFA in form of ethyl esters. It is stabilized with mixed tocopherols, ascorbyl palmitate, citric acid and contains rosemary extract.

1. A method for determining the seriousness of side effects to be expected in humans after consumption of foods or nutritional supplements containing oils which method is characterized by analyzing the food or nutritional supplement oils using the FAST Index™ methodology and evaluating the result with the Fish Taste algorithm.

2. The method of claim 1 wherein the oils are rich in polyunsaturated fatty acids (PUFAs).

3. A method for predicting the acceptance of foods or nutritional supplements containing oils by consumers which method is characterized by analyzing the food or nutritional supplement oils using the FAST Index™ methodology and evaluating the result with the Fish Taste algorithm.

4. The method of claim 3 wherein the oils are rich in PUFAs.

5. The use of the FAST Index™ for predicting (a) the seriousness of side effects to be expected in humans after consumption of foods or nutritional supplements containing oils and/or (b) the acceptance of such foods or nutritional supplements by consumers.

6. The use of the FAST Index™ according to claim 5 wherein the oils are rich in PUFAs.
